(19) United States
(12) Patent Application Publication
(10) Pub. No.: US 2021/0355159 A1
(43) Pub. Date: Nov. 18, 2021

(54) IN-LINE PRODUCT CONCENTRATION TO REDUCE VOLUMETRIC LOAD FLOW RATE AND INCREASE PRODUCTIVITY OF A BIND AND ELUTE CHROMATOGRAPHY PURIFICATION

(71) Applicant: EMD Millipore Corporation, Burlington, MA (US)

(72) Inventors: Thomas Elich, Arlington, MA (US); Herbert Lutz, Playa del Ray, CA (US)

(21) Appl. No.: 17/311,099

(22) PCT Filed: Nov. 11, 2019
(86) PCT No.: PCT/US2019/060676

§ 371 Date: Jun. 4, 2021

Related U.S. Application Data
(60) Provisional application No. 62/782,671, filed on Dec. 20, 2018.

(51) Int. Cl. C07K 1/22; C07K 1/36; C07K 1/34

(52) U.S. Cl. CPC C07K 1/22; C07K 1/34; C07K 1/36

(57) ABSTRACT

Method and system for purifying a sample comprising a biomolecule of interest and impurities, comprising expressing said biomolecule of interest in a bioreactor to form a product sample comprising said biomolecule of interest and impurities; subjecting said product sample to single pass tangential flow filtration to form a concentrated product sample; and subjecting said concentrated product sample to affinity chromatography to remove impurities from said concentrated product sample.

### CROSS-REFERENCE TO RELATED APPLICATIONS

The present application claims the benefit of priority of U.S. Provisional Application No. 62/782,671, filed Dec. 20, 2018, the entire contents of which is incorporated by reference herein in its entirety.

### FIELD

The present disclosure relates to efficient processes and systems for the purification of biological molecules including therapeutic antibodies and Fc-containing proteins.

### BACKGROUND

The general process for the manufacture of biomolecules, such as proteins, particularly recombinant proteins, typically involves two main steps: (1) the expression of the protein in a host cell, followed by (2) the purification of the protein. The first step involves growing the desired host cell in a bioreactor to effect the expression of the protein. Some examples of cell lines used for this purpose include Chinese hamster ovary (CHO) cells, myeloma (NSO) bacterial cells such as e-coli and insect cells. Once the protein is expressed at the desired levels, the protein is removed from the host cell and harvested. Suspended particulates, such as cells, cell fragments, lipids and other insoluble matter are typically removed from the protein-containing fluid in a downstream purification process, resulting in a clarified fluid containing the protein of interest in solution as well as other soluble impurities.

The second step involves the purification of the harvested protein to remove impurities that are inherent to the process. The main goal of the harvest and downstream operations is to isolate the product (e.g., expressed protein) from the soluble/insoluble impurities. Examples of impurities include host cell proteins (HCP, proteins other than the desired or targeted protein), nucleic acids, endotoxins, viruses, protein variants, protein aggregates, and cell culture media components/additives. This purification typically involves several chromatography steps, which can include one or more of affinity chromatography, cation-exchange chromatography in bind/elute mode, anion-exchange chromatography in flow through mode, hydrophobic interaction, etc. on solid matrices such as porous agarose, polymeric or glass or by membrane based adsorbers.

One example of a process template includes primary clarification by centrifugation, secondary clarification by filtration, and a chromatography process train involving protein-A affinity in bind/elute mode, followed by cation exchange in bind/elute mode, followed by anion exchange in flow through mode. The protein-A column captures the protein of interest or target protein by an affinity mechanism while the bulk of the impurities pass through the column to be discarded. The protein then is recovered by elution from the column. Since most of the proteins of interest have isoelectric points (PI) in the basic range (8-9) and therefore being positively charged under normal processing conditions (pH below the PI of the protein), they are bound to the cation exchange resin in the second column. Other positively charged impurities are also bound to this resin. The protein of interest is then recovered by elution from this column under conditions (pH, salt concentration) in which the protein elutes while the impurities remain bound to the resin. The anion exchange column is typically operated in a flow through mode, such that any negatively charged impurities are bound to the resin while the positively charged protein of interest is recovered in the flow through stream. Following the downstream purification process, ultrafiltration/diafiltration can be used to condition the buffer system and to concentrate the product prior the final fill unit operation to complete the manufacturing process. This process results in a highly purified and concentrated protein solution, which can be critical particularly where the therapeutic proteins are meant for use in humans and have to be approved by regulatory agencies, such as the Food and Drug Administration (FDA).

FIG. 3 illustrates one conventional process. The process includes a cell harvest step, which may involve use of centrifugation to remove cell and cell debris from a cell culture broth, followed by depth filtration. The cell harvest step is usually followed by a capture step such as a Protein A affinity purification step, which is followed by virus inactivation. Virus inactivation is typically followed by one or more chromatographic steps, also referred to as polishing steps, which usually include one or more of cation exchange chromatography and/or anion exchange chromatography and/or hydrophobic interaction chromatography and/or mixed mode chromatography and/or hydroxyapatite chromatography. The polishing steps are followed by virus filtration and ultrafiltration/diafiltration, which completes the process.

The capture step may use Protein-A resin such as Eshmuno® A resin, a rigid, Protein A affinity chromatography resin or ProSep® Ultra Plus media, both commercially available from EMD Millipore Corporation, especially for antibodies containing Fc regions. Other resins operated in bind and elute mode also may be suitable for capture. Bind and elute chromatography includes (1) product loading to a target binding capacity, (2) elution of product from the column, and (3) cleaning to prepare the resin for re-use.

The relatively low binding capacity, coupled with the high costs associated with chromatography resins suitable for this application, require manufacturers to perform numerous bind/elute and column regeneration cycles using the chromatography media in order to make such processes cost-effective. The regeneration processes further increase production costs due to decreased product throughput, increased consumption of buffers and cleaning agents, validation costs, and increased capital equipment requirements.

There is an important relationship between the dynamic binding capacity (DBC) and volumetric load flow rate of the chromatography resin, as shown in FIG. 1. The DBC is defined as the mass of product bound to the resin at a given loading. The volumetric flow rate is inversely proportional to residence time (the amount of time it takes for an unretained molecule to travel through the column). At lower flow rates, there is more time for target molecules to diffuse into the resin pore structure, and thus the DBC is typically higher. At higher flow rates, a significant portion of the loaded product flows through unbound, and the DBC is lower. FIG. 2 plots the percent of unbound product breakthrough as a function of mass loading for a range of volumetric flows.

Protein binding capacity is an important parameter for chromatography media; it determines the amount of media required to purify a given amount of protein. Ideally, each chromatography column would be operated at a DBC near the static binding capacity (SBC) of the resin, which is the maximum capacity of the resin. The SBC of a resin is independent of flow rate. The ratio of DBC to SBC is the resin utilization.

It can be seen from FIGS. 1 and 2 that it is preferred to operate the capture column(s) at low volumetric flow rate(s). However, low volumetric flow operation is not always practical, as it would result in a corresponding reduction in mass flow rate and diminished productivity (defined as the mass processed per volume of chromatography resin and process time).

In a multi-column capture approach, a rotation of 2-6 identical columns may be employed such that at least 1 column is always available for product loading (while elution and regeneration occur in the other column or columns). To maximize resin utilization, two or more columns may be loaded in series. The serial loading approach is used to capture unbound product breakthrough from the first column in the series, thereby maximizing yield.

In current methods for continuous bioprocessing, the upstream bioreactor is linked directly to the multi-column capture step, and the flow rates of the two steps must be harmonized. The bioreactor harvest rate typically dictates the process rate for subsequent steps, as reducing the bioreactor production rate is not a practical option. To maintain high Protein A binding capacity in conventional operation, it is common to use relatively large column volumes so as to increase residence time at a given volumetric flow rate, and/or increase the number of columns being cycled. However, increasing the number of columns or column size negatively impacts process productivity.

It would therefore be desirable to optimize capture chromatography in continuous bioprocessing operations without deleteriously impacting productivity. It also would be desirable to provide a system and process for the manufacture of biomolecules such as monoclonal antibodies (Mabs) that is efficient and cost-effective.

### SUMMARY

The problems of the prior art have been overcome by the embodiments disclosed herein, which provides a method and system for enabling low volumetric flow chromatography loading in a biomanufacturing process without affecting the product mass flow rate. In certain embodiments, in-line product concentration is carried out, such as by single pass tangential flow filtration, upstream of capture chromatography. The addition of in-line concentration does not impact the alloted process time, allowing the chromatography step to be operated at a lower volumetric flow rate (less volume to process in the same amount of time). Moreover, since in-line concentration simultaneously increases product concentration while proportionally decreasing volume, the product mass flow rate is unchanged. As seen in FIGS. 1 and 2, this low volumetric flow operation increases the DBC at a target loading while decreasing the amount of unbound product breakthrough. The result is more efficient utilization of the chromatography resin.

Embodiments disclosed herein include purification and isolation of biomolecules of interest derived from cell culture fluids. In certain embodiments, methods and systems disclosed include in-line concentration followed by a downstream purification process. In certain embodiments, the downstream purification process may include sequential purification by one or more chromatography columns.

In certain embodiments, a method for purifying a sample comprising a biomolecule of interest and impurities is disclosed, the method comprising expressing the biomolecule of interest in a bioreactor to form a product sample comprising the biomolecule of interest and impurities; subjecting the product sample to single pass tangential flow filtration to form a concentrated product sample; and subjecting the concentrated product sample to affinity chromatography to remove impurities from it. In certain embodiments, the product sample (e.g., harvested cell culture) from the biorector is subjected to one or more clarification steps prior to being subjected to the single pass tangential flow filtration. The one or more clarification steps may include one or more of centrifugation, tangential flow filtration, depth filtration, and sterile filtration. In certain embodiments, the concentrated product sample exiting the singe pass tangential flow filtration operation is passed through a one or more sterile filters, or enters a tank, prior to being subjected to affinity chromatography.

In certain embodiments, the affinity chromatography uses Protein A affinity ligand.

In certain embodiments, the method further comprises subjecting the concentrated product sample to a virus inactivation step.

In certain embodiments, the method comprises subjecting the concentrated product sample to a polishing step downstream of capture by affinity chromatography. In some embodiments, the polishing step comprises one or more of anion exchange chromatography, cation exchange chromatography, and hydrophobic interaction chromatography. In some embodiments, both virus activation and polishing may be carried out.

In certain embodiments, the biomolecule is an antibody selected from the group consisting of a recombinant antibody, a recombinant monoclonal antibody, a polyclonal antibody, a humanized antibody and an antibody fragment. In some embodiments, the biomolecule is a protein.

In certain embodiments, a system for purifying a biomolecule of interest is disclosed, the system comprising a bioreactor; an in-line single pass tangential flow filtration unit downstream of the bioreactor for continuously concentrating the product sample exiting from the bioreactor; at least two affinity chromatography columns configured in series downstream of the in-line single pass tangential flow filtration unit for receiving the concentrated product stream from the in-line single pass tangential flow filtration unit; a virus inactivation filter positioned downstream of the at least two affinity chromatogrpahy columns; and one or more anion exchange, cation exchange or hydrophobic interaction exchange chromatography columns positioned downstream of the virus inactivation filter.

In some embodiments, the system includes one or more of a centrifuge, tangential flow filtration unit, depth filtration unit, and sterile filtration unit downstream of the bioreactor and upstream of the SPTFF unit. In some embodiments, the system includes one or more sterile filters and/or one or more tanks or vessels downstream of the SPTFF unit and upstream of the affinity chromatography columns.

In some embodiments, the at least two affinity chromatography columns each comprise Protein A affinity ligand. In some embodiments, there are exactly two affinity chromatography columns.

### BRIEF DESCRIPTION OF THE DRAWINGS

FIG. 1 is a graph of mass loading vs. dynamic binding capacity (DBC) at different feed flow rates;

FIG. 2 is graph of mass loading vs. percent unbound prduct breakthrough at different feed flow rates;

FIG. 3 is a schematic representation of a conventional purification process used in the industry; and

FIG. 4 is a schematic representation of a purification system in accordance with certain embodiments.

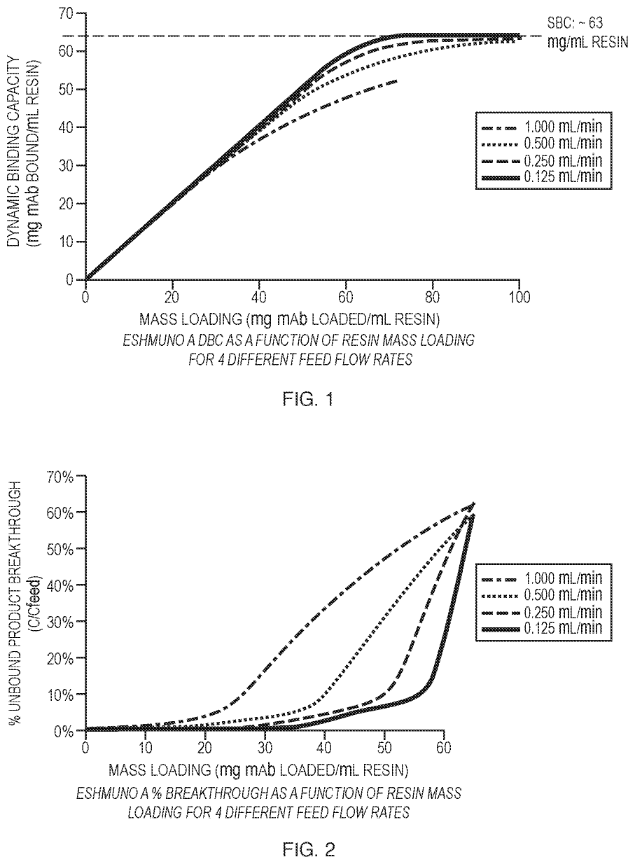

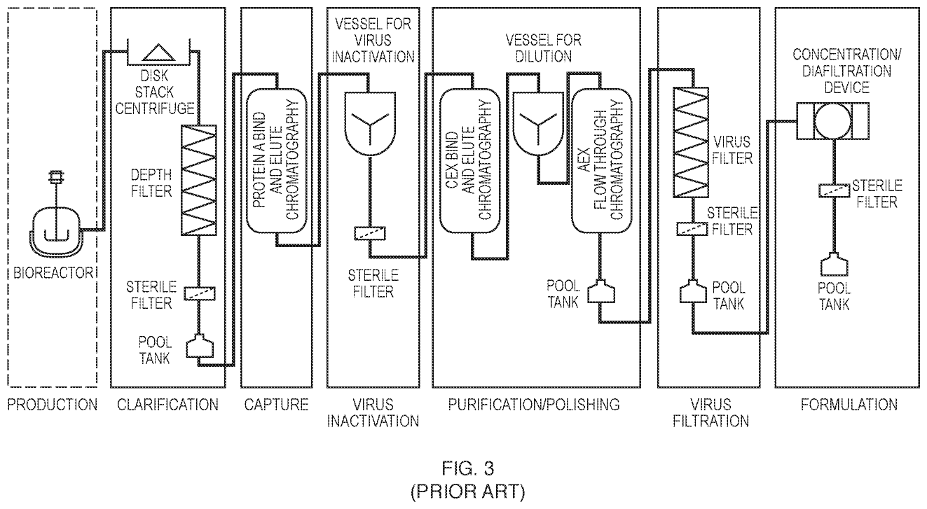

### DETAILED DESCRIPTION

In the following description, the terms “selected biomolecule”, “target biomolecule” or “molecule”, “target protein”, “biomolecule or protein of interest”, or similar terms all refer to products of a biomolecule manufacturing process.

The terms “contaminant,” “impurity,” and “debris,” as may be used interchangeably herein, refer to any foreign or objectionable molecule, including a biological macromolecule such as a DNA, an RNA, one or more host cell proteins, endotoxins, lipids, protein aggregates and one or more additives which may be present in a sample containing the product of interest that is being separated from one or more of the foreign or objectionable molecules. Additionally, such a contaminant may include any reagent which is used in a step which may occur prior to the separation process.

As used herein, the term “sample” refers to any composition or mixture that contains a target molecule, such as a target protein, to be purified. Samples may be derived from biological or other sources. Biological sources include eukaryotic and prokaryotic sources, such as plant and animal cells, tissues and organs. In some embodiments, a sample includes a biopharmaceutical preparation containing a protein of interest to be purified. In a particular embodiment, the sample is a cell culture feed containing a protein of interest to be purified. The sample may also include diluents, buffers, detergents, and contaminating species, debris and the like that are found mixed with the target protein or protein of interest. The sample may be “partially purified” (i.e., having been subjected to one or more purification steps, such as filtration steps) or may be obtained directly from a host cell or organism producing the target molecule (e.g., the sample may comprise harvested cell culture fluid).

The terms “bind and elute mode” and “bind and elute process,” as used herein, refer to a separation technique in which at least one target molecule contained in a sample (e.g., an Fc region containing protein) binds to a suitable resin or media (e.g., an affinity chromatography media or a cation exchange chromatography media) and is subsequently eluted.

The term “break-through,” as used herein, refers to the point of time during the loading of a sample containing a target molecule onto a packed chromatography column or separation unit, when the target molecule first appears in the output from the column or separation unit. In other words, the term “break-through” is the point of time when loss of target molecule begins.

As used herein, the phrase “consisting essentially of” limits the scope of a claim to the specified materials or steps and those that do not materially affect the basic and novel characteristics of the claimed subject matter. The term permits the inclusion of elements or steps which do not materially affect the basic and novel characteristics of the apparatus, system or method under consideration. Accordingly, the expressions “consists essentially of” or “consisting essentially of” mean that the recited embodiment, feature, component, step, etc. must be present and that other embodiments, features, components, steps, etc., may be present provided the presence thereof does not materially affect the performance, character or effect of the recited embodiment, feature, component, step, etc. The presence of an operation or step that has no material effect on the sample or product is permitted. For example, a method consisting essentially of purifying a sample comprising a biomolecule of interest and impurities, consisting essentially of expressing said biomoelcule of interest in a bioreactor to form a product sample comprisng said biomolecule of interest and impurities; clarifying the product sample; subjecting said clarified product sample to single pass tangential flow filtration to form a concentrated product sample; and subjecting said concentrated product sample to affinity chromatography to remove impurities from said concentrated product sample, excludes other steps or unit operations carried out between the clarification and the single pass tangential flow filtration operations, and excludes other steps or unit operations carried out between the single pass tangential flow filration and affinity chromatography operations, that would materially change the composition of the product sample. A sterile filtration step or a tank or vessel positioned downstream of the SPTFF unit and upstream of capture chromatography would not materially change the composition of the product sample.

In certain embodiments, the sample that is the starting material of the process may vary depending upon the cell line in which it was grown as well as the conditions under which it is grown and harvested. For example, in most CHO cell processes the cells express the molecule outside of the cell wall into the media. One tries not to rupture the cells during harvest in order to reduce the amount impurities in the mixture. However, some cells during growth and harvesting may rupture due to shear or other handling conditions or die and lyse, spilling their contents into the mixture. In bacteria cell systems, the biomolecule is often kept with the cellular wall or it may actually be part of the cellular wall (Protein A). In these systems the cell walls need to be disrupted or lysed in order to recover the biomolecule of interest.

The target molecule to be purified can be any biomolecule, preferably a protein, in particular, recombinant protein produced in any host cell, including but not limited to, Chinese hamster ovary (CHO) cells, Per.C6® cell lines available from Crucell of the Netherlands, myeloma cells such as NSO cells, other animal cells such as mouse cells, insect cells, or microbial cells such as E. coli or yeast. Additionally, the mixture may be a fluid derived from an animal modified to produce a transgenic fluid such as milk or blood that contains the biomolecule of interest. Optimal target proteins are antibodies, immunoadhesins and other antibody-like molecules, such as fusion proteins including a C_{H}2/C_{H}3 region. For example, this product and process can be used for purification of recombinant humanized monoclonal antibodies such as (RhuMAb) from a conditioned harvested cell culture fluid (HCCF) grown in Chinese hamster ovary (CHO) cells expressing RhuMAb.

In certain embodiments, in the downstream purification process, a series of purification media having the desired chemical functionalities are used to effect the removal of soluble impurities while the product remains in solution and flows through the purification media, resulting in a purified stream containing the product. Suitable forms of purification media include derivatized membranes, functionalized chromatography media, or any other porous material having the desired chemical functionality to interact with the various impurities so that the media can capture the impurities by electrostatic, hydrophobic, or affinity interactions. In view of the complex and varied natured of the impurities, a multitude of purification media having different chemical functionalities can be arranged in series to remove a variety of impurities having different chemical properties.

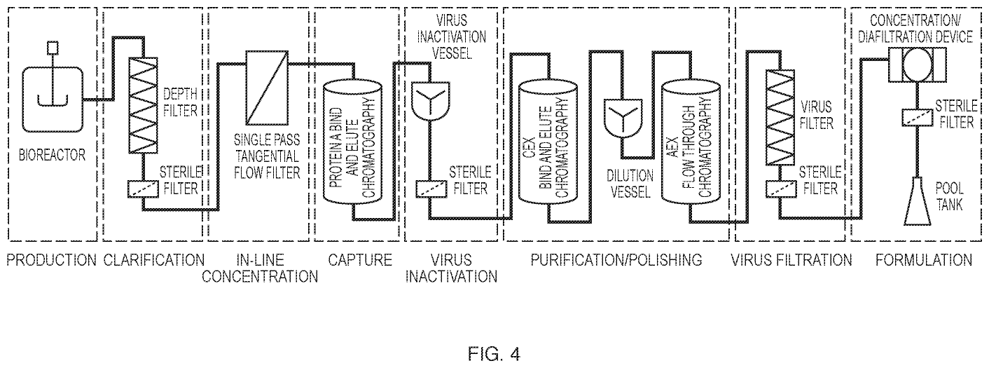

In certain embodiments, disclosed is a process for purifying a target molecule from a sample, where the process comprises: (a) expressing a protein in a bioreactor to form a protein sample; (b) subjecting the protein sample to an in-line concentration step to form a concentrated protein sample; (c) subjecting the resulting concentrated protein sample to Protein A affinity chromatography, which employs one or more affinity chromatography units. In certain embodiments, the protein sample is subjected to one or more clarification steps prior to being subjected to an in-line concentration step. Also disclosed as illustrated in FIG. 4 is a system for purifying a target molecule from a sample, comprising a bioreactor, an in-line single pass tangential flow filter, and one or more Protein A affinity chromatography columns in fluid communication with the in-line single pass tangential flow filter. In certain embodiments, the system may include one or more of a centrifuge, a tangential flow filter, a depth filter, and a sterile filter downstream of the bioreactor and upstream of the single pass tangential flow filter. In some embodiments, the system may include one or more of a sterile filter and a tank or vessel downstream of the single pass tangential flow filter and upstream of the affinity chromotography columns. The tank or vessel may be a surge tank or vessel. In some embodiments, one or more virus inactivation units may be downstream of the affinity chromatography columns. In some embodiments, a polishing phase may be downstream of the Protein A affinity chromatography columns, and may include one or more of an anion exchange chromatography column, a cation exchange chromatography column, and a hydrophobic interaction chromatography column. In some embodiments, the system may include one or more of a virus filter, a sterile filter, and concentration/diafiltration device downstream of the polishing phase.

In some embodiments, there is a connecting line between the various devices in the system. The devices are connected in line such that each device in the system is in fluid communication with devices that precede and follow the device in the system. In certain embodiments, a single pass tangential flow filtration unit is immediately downstream of one or more clarification units, such as a centrifuge, a tangential flow filtration unit (e.g., one or more TFF modules), a depth filtration unit such as a Clarisolve® depth filter commercially available from MilliporeSigma, and/or a sterile filtration unit (e.g., a sterile filtration membrane), with no unit operation carried out between the final clarification step and the single pass tangential flow filtration. In certain embodiments, the single pass tangential flow filtration unit is immediately upstream of the affinity chromatography column or columns, which is preferably a Protein A column or columns, with no unit operation carried out in between. In other embodiments, the single pass tangential flow filtration unit is immediately upstream of a sterile filtration membrane and/or a tank, and the sterile filtration membrane and/or tank is immediately upstream of the affinity chromatography column or columns, which is preferably a Protein A column or columns. The tank may be used as a surge vessel to protect against pump variability between unit operations in a continuous process, or to provide an operator sampling point for the process intermediate pool. The tank may also be used as a surge vessel to enable single column operation as discussed in greater detail below. The tank could also be used as a product hold vessel, for example in the case of a batch-wise upstream process connected to a continuous downstream process. In some embodiments, the bioreactor used in a system according to the present invention is a disposable or a single use bioreactor. In some embodiments, the system is enclosed in a sterile environment.

In some embodiments, the starting sample is a cell culture. Such a sample may be provided in a bioreactor. In certain embodiments, the bioreactor is a perfusion bioreactor.

In accordance with certain embodiments, in-line product concentration removes excess water and buffer from the feed, thereby reducing the volume loaded to the chromatography column or columns downstream of the in-line concentration.

In some embodiments, the bind and elute chromatography apparatus includes at least two separation units, with each unit comprising the same chromatography media, e.g., Protein A affinity media. In a particular embodiment, the Protein A media comprises a Protein A ligand coupled to a rigid hydrophilic polyvinylether polymer matrix. In other embodiments, the Protein A ligand may be coupled to agarose or controlled pore glass. The Protein A ligand may be based on a naturally occurring domain of Protein A from Staphylococcus aureus or be a variant or a fragment of a naturally occurring domain. In a particular embodiment, the Protein A ligand is derived from the C domain of Staphylococcus aureus Protein A. The separation units are connected to be in fluid communication with each other in series, such that a liquid can flow from one separation unit to the next.

In other embodiments, the bind and elute chromatography apparatus includes at least three separation units. The separation units are connected to be in fluid communication with each other in series, such that a liquid can flow from one separation unit to the next. In some embodiments, the bind and elute chromatography apparatus includes at least four separation units, or at least five separation units, or at least six separation units, connected to be in fluid communication with each other in series.

Single pass tangential flow filtration (SPTFF) allows for sufficient concentration of product in a single pass through the filter assembly in a continuous mode, and thus does not require retentate return and multiple passes through the filter in batch mode. This may be made possible by increasing the fluid residence time in membrane channels in the device via lower feed flux and/or longer channels compared to multiple pass TFF. Further advantages include the ability to use constant feed flow and retentate pressure, and smaller pumps and a smaller facility footprint. Suitable membranes for SPTFF include ultrafiltration membranes ranging from 1-1000 kD nominal molecular weight limit. For example, Pellicon® 2 or Pellicon® 3 cassettes commercially available from MilliporeSigma can be used. A single cassette may be used, or multiple cassettes arranged in series to improve conversion may be used. Since the tangential flow filtration step sufficiently concentrates the product sample, a retentate recycle is not required. In certain embodiments, the SPTFF accomplishes increased sample residence time by configuring TFF cassettes in series.

In certain embodiments, the SPTFF device is positioned immediately upstream of a capture chromatography column, such as a Protein A chromatography column. In other embodiments, a sterile filter and/or a tank may be positioned between the SPTFF device and the capture chromatography column. In certain embodiments, the SPTFF device is placed immediately downstream of a harvested cell culture clarification unit operation, such as one or more centrifuges, tangential filtration modules, depth filtration unit, or sterile filtration unit. The pre-concentration of the biomolecule of interest using an SPTFF device reduces the overall process volume from the bioreactor that proceeds to the capture chromatography step, without changing the overall process time or the product mass flow rate.

In certain embodiments, the inclusion of a single pass tangential flow filtration unit upstream of affinity chromatography reduces the number of affinity chromatography columns that must be loaded in series to maintain the desired yield. Since the volumetric flow rate is reduced, the affinity chromatography media (e.g., containing Protein A affinity ligand) reaches the target dynamic binding capacity at lower mass loadings with minimal breakthrough, thereby allowing a reduction in the number of columns that otherwise would be required. In certain embodiments, only two affinity chromatography columns (e.g., containing Protein A affinity ligand) in series are needed. In some embodiments, where a surge vessel or the like is used to collect load material during column wash, elution and cleaning, only a single affinity chromatography (e.g., containing Protein A affinity ligand) column is required.

When fewer columns are used, the columns must be cycled more frequently during processing, which makes it easier for the end user to realize complete lifetime and cost of the chromatography media. Reducing the number of cycled columns also lowers system pressures, such as a reduction of 25%, and decreases the system size and complexity; fewer valves and valve switching are required.

The use of the single pass tangential flow filtration device also enables more efficient utilization of the chromatography resin, and thus less resin is required to process the same amount of material in the same amount of time, improving productivity and reducing cost. A reduced volumetric flow rate lessens the burden on the pumps in the system, enabling smaller systems capable of processing more product mass.

1. A method for purifying a sample comprising a biomolecule of interest and impurities, comprising expressing said biomolecule of interest in a bioreactor to form a product sample comprising said biomolecule of interest and impurities; subjecting said product sample to a clarification operation, subjecting the resulting clarified product to a single pass tangential flow filtration to form a concentrated product sample; and subjecting said concentrated product sample to affinity chromatography to remove impurities from said concentrated product sample.

2. The method of claim 1, wherein said affinity chromatography comprises Protein A affinity ligand.

3. The method of claim 1, further comprising subjecting said concentrated product sample to a virus inactivation step downstream of said affinity chromatography.

4. The method of claim 3, further comprising subjecting said concentrated product sample to a polishing step downstream of said virus inactivation step.

5. The method of claim 4, wherein said polishing step comprising one or more of anion exchange chromatography, cation exchange chromatography, and hydrophobic interaction chromatography.

6. The method of claim 1, wherein the biomolecule is an antibody selected from the group consisting of a recombinant antibody, a recombinant monoclonal antibody, a polyclonal antibody, a humanized antibody and an antibody fragment.

7. The method of claim 1, wherein said biomolecule is a protein.

8. The method of claim 1, further comprising subjecting said concentrated product sample to sterile filtration prior to subjecting it to affinity chromatography.

9. A system for purifying a biomolecule of interest, comprising:
a. a bioreactor;
b. an in-line single pass tangential flow filtration unit downstream of said bioreactor for continuously concentrating the product sample exiting from said bioreactor;
c. at least two affinity chromatography columns configured in series downstream of said in-line single pass tangential flow filtration unit for receiving the concentrated product stream from said in-line single pass tangential flow filtration unit;
d. a virus inactivation filter positioned downstream of said at least two affinity chromatography columns; and
e. one or more anion exchange, cation exchange or hydrophobic interaction exchange chromatography columns positioned downstream of said virus inactivation filter.

10. The system of claim 9, wherein said at least two affinity chromatography columns each comprise Protein A affinity ligand.

11. The system of claim 9, further comprising a sterile filter positioned between said bioreactor and said single pass tangential flow filtration unit.

12. A method of purifying a sample comprising a biomolecule of interest and impurities, consisting essentially of expressing said biomolecule of interest in a bioreactor to form a product sample comprising said biomolecule of interest and impurities; subjecting said product sample to single pass tangential flow filtration to form a concentrated product sample; and subjecting said concentrated product sample to affinity chromatography to remove impurities from said concentrated product sample.
